(19) United States
(12) Patent Application Publication
(10) Pub. No.: US 2004/0226055 A1
(43) Pub. Date: Nov. 11, 2004

(54) Animal model exhibiting pathological conditions of Alzheimer's disease

(76) Inventor: Tadeusz Wieloch, Lund (SE)

Correspondence Address: BOZICEVIC, FIELD & FRANCIS LLP, 1900 University Ave, East Palo Altho, CA, 94303, US

(21) Appl. No.: 10/764,292

(22) Filed: Jan. 22, 2004

Related U.S. Application Data
(60) Provisional application No. 60/442,568, filed on Jan. 23, 2003.

(51) Int. Cl. C12Q 1/68; G01N 33/53; G01N 33/567; A61K 49/00

(52) U.S. Cl. 800/12; 424/9.2; 435/6; 435/7.2

(57) ABSTRACT

This invention provides an animal model that exhibits a pathological condition characteristic of Alzheimer's disease. This invention also provides various methods of using this animal model for the development of modulators of Alzheimer's disease-associated genes or polypeptides. Further provided in the present invention is a method of developing a modulator of pathogenesis of Alzheimer's disease. The compositions and methods of the present invention are particularly useful in Alzheimer's disease research and drug development.

### CROSS-REFERENCE TO RELATED APPLICATIONS

This application claims the priority benefit of U.S. Provisional Patent Application 60/442,568 filed Jan. 23, 2003, pending, which is hereby incorporated herein by reference in its entirety.

### TECHNICAL FIELD

This invention is in the field of Alzheimer's disease research. Specifically, the invention relates to the generation of an animal model that exhibits a pathological condition of Alzheimer's disease. The compositions and methods embodied in the present invention are particularly useful for the development of diagnosis and/or therapeutics of Alzheimer's disease.

### BACKGROUND OF THE INVENTION

Alzheimer's Disease (AD) is the most common progressive dementia in the elderly for which there is no cure or effective therapy. To date, more than 15 million people have been diagnosed with AD. Approximately 10% of the population over 65 is expected to develop AD, and nearly half of all people over age 85 are afflicted with this disease. In the United States, AD is the fourth leading cause of death of the elderly, imposing an enormous cost to the society.

AD selectively affects neurons in certain brain regions and neural systems. It causes dysfunction and death of vulnerable populations of neuronal cells in the cortex, hippocampus, amygdala, anterior thalamus, basal forebrain, and several brainstem monoaminergic nuclei. The progressive deterioration of certain brain regions and neuronal cells manifests with memory failure, disorientation, and confusion.

The principal neuropathological hallmark of AD is the progressive formation of insoluble amyloid plaques and vascular deposits consisting of the 4 kDa beta amyloid peptide (AB peptide). AB peptide occurs in two predominant forms with different C-termini, Aβ1-40 and Aβ1-42 which have been suggested to cause the familial early-onset Alzheimer's disease. Formation of Aβ requires proteolytic cleavage of a large transmembrane protein, the β-amyloid precursor proteins (BAPP) by one or more secretases, namely beta-secretase (β-secretase) and gamma-secretase (γ-secretase). See, e.g., reviewed in Selkoe(1999) Nature 399:A23-31; Yankner (2000) Ann. N.Y. Acad. Sci. 924:26-8; Tandon et al.(2000) Current Opinion Neurol. 13(4):377-84. Briefly, to initiate Aβ formation, beta-secretase (β-secretase) cleaves βAPP at the N-terminus to release a soluble N-terminal fragment (APPsβ), and a C-terminal fragment (C99) which remain membrane bound. In an alternative pathway, α-secretase produces a large soluble N-terminal fragment (APPsα) and a 10 kDa membrane-bound C-terminal fragment (C83). Both C99 and C83 can be further cleaved by one or more γ-secretase, yielding the Aβ peptide. Recent studies have shown that overexpression of β-secretase in cell culture leads to more production of APPₛβ (Vassar, et al. (1999) Science 286:735-741). Some genetic changes (e.g. mutations in βAPP) and age related changes in the metabolism of the brain (e.g. decreased energy supply and increased oxidative ROS) are also in favor of production of this neurodegenerative peptide. Mattson, et al. (1993) Trends Neurosci 16:409-414; Mattson, et al. (1997) Physiol Rev 77:1081-1132. Both genetic and biochemical studies strongly implicate that deposition of Aβ plaques is ultimately responsible for the neuronal damage and death that underlie AD dementia.

Despite the increasing knowledge on the underlying genetic attributes, the molecular basis of AD pathogenesis is far from being fully elucidated. AD research has traditionally been hampered by the lack of an efficient and cost effective method to generate suitable animal models. Current AD models are primarily transgenic animals carrying one or more mutant genes implicated in Aβ formation. These transgenic animals, however, are generated through a laborious and time-consuming process. Moreover, not all models display pathological conditions characteristic of AD. For those that do exhibit AD-like pathology, such conditions may have taken a number of months and even years to develop. For instance, even a “bigenic” mice designated hAPP^{swe}×hPS1^{ΔE9}, that carry double mutations requires 8 months to develop initial Aβ deposits (Borchelt et al. (1997) Neuron 19: 939-945). See also Sturchler-Pierrat et al. Proc. Natl. Sci. USA (1997) 94:13287-13292; Chapman et al. (1999) Nature Neuroscience 2(3): 271-276). Thus, there remains a considerable need for methods of generating animal models that would reproducibly exhibit an AD pathological condition in an accelerated fashion.

Recent studies suggest that chronic brain pathological changes share some similarity with AD pathogenesis. It has been shown that delayed damages occur after an acute neural degeneration in both experimental and clinical settings. For instance, following an early phrase of cellular degeneration that occurs in brain structures within minutes to hours from a transient ischemic insult, a delayed phase of neural death develops between 48 to 72 hours (Kirino, et al., (1984) Acta Neuropathol 64:139-147; Siesjo, B. K. (1992) J Neurosurg 77:169-184; Lee, et al. (2000) J Clin Invest 106:723-731; Dimagl, et al. (1999) Trends Neurosci 22:391-397; Minamisawa, et al. (1990) Ann Neurol 28:26-33). Dietrich and co-workers have reported that significant damage to the dorsolateral striatum can be seen at two months, but not at three days after the ischemic insult (Dietrich, et al. (1995) J Cereb Blood Flow Metab 15:960-968). In another study it was shown that a forebrain ischemic insult of ten minutes resulted in striatal changes involving both neuronal and glial elements, which became evident after two to four weeks of survival or longer (Lin, et al. (1998) Acta Neuropathol 95:511-523). In that report, an increased number of reactive astrocytes and proliferated microglia was also seen throughout the eight post ischemic weeks, with a tendency towards subsidence at ten weeks. More recent studies have revealed that upon induction of a transient forebrain ischemia, β-amyloid precursor protein (βAPP) is overexpressed in regions with necrotic cell death for example CA1 of hippocampus, striatum and thalamus (Lin, et al. (1999) Acta Neuropathol 97:359-368). However, these studies do not show or suggest whether animals suffering from a transient forebrain ischemia develop a pathological condition characteristic of AD, e.g., the accumulation of Aβ peptide in the brain tissue.

### SUMMARY OF THE INVENTION

The present invention provides a method for generating an animal model exhibiting an Alzheimer's disease pathological condition. Unlike the conventional transgenic AD models, the subject animal model reproducibly exhibits pathological condition characteristic of AD in an accelerated manner.

The subject method involves (a) inducing a transient forebrain ischemia in the animal; and (b) allowing the animal to recover from the ischemic induction for a sufficient amount of time so that a pathological condition of AD is exhibited. An exemplary pathological condition is characterized by differential expression of an AD-associated gene or polypeptide, preferably the overproduction of Aβ peptide or β-secretase. The detection of differential expression of a gene or protein can be carried out in a hybridization assay and immunoassay, respectively. Suitable animal models can be selected from the group consisting of mammal, primate, and rodent. Preferred models include but are not limited to rat, mouse, guinea pig, dog, cat, rabbit, pig, chimpanzee, and monkey. Older animals are more preferred over young animals. The ischemic insult may last for more than 10 minutes, preferably between about 15 to about 20 minutes. Upon induction of an ischemic insult, the animals are allowed to recover for least about 2 weeks, preferably for at least about 4 weeks, more preferably for about 4 weeks to about 10 weeks.

The present invention provides an animal model generated by the aforementioned method.

The invention also provides a method of developing a modulator of pathogenesis of AD. The method comprises the steps of: (a) administering a candidate modulator to a test animal model generated by a method comprising (i) inducing a transient and reversible forebrain ischemia in the animal; and (ii) allowing the animal to recover from the ischemic induction for a sufficient amount of time so that a pathological condition of AD is exhibited; and (b) detecting a change in the pathological condition in the test animal model of (a) relative to a control. In one aspect, the modulator so developed is capable of ameliorating a pathological condition of AD. In another aspect, the modulator is capable of advancing a pathological condition of AD. The detected pathological conditions include differential expression of an AD-associated gene or polypeptide (e.g., accumulation of Aβ peptide or β-secretase), beta-amyloid plaque formation, plaque-induced mononuclear phagocyte activation, plaque-induced mononuclear phagocyte neurotoxicity, and neuronal loss within the brain. Candidate modulator is selected from the group consisting of an antisense oligonucleotide, a ribozyme, a ribozyme derivative, an antibody, a liposome, a small interfering RNA, a small molecule and an inorganic compound. The candidate modulator can be administered to the test animal model intravenously, subcutaneously, intramuscularly, intraperitoneally, intradermally, orally, intranasally, or intrapulmonarily.

Further provided in the present invention is a method of developing a modulator of an AD-associated gene or protein. The method comprises: (a) contacting a candidate modulator with an AD-associated gene or protein that is contained in a test biological sample derived from an animal model, wherein the animal model is generated by a method comprising: (i) inducing a transient forebrain ischemia in the animal; and (ii) allowing the animal to recover from the ischemic induction for a sufficient amount of time so that a pathological condition of AD is exhibited; and (b) detecting an alteration in expression of the AD-associated gene or protein, or an alteration in activity of the protein of step (a), relative to a control sample.

### BRIEF DESCRIPTION OF THE DRAWINGS

FIG. 1 depicts sections magnified 200×, stained with Celestine blue and acid fuchsin. In the hippocampus of sham operated animals (A), the staining shows normal morphology with violet stained neurons, rounded cells and large nuclei. In the ischemic animals at four weeks of recovery (B) and ten weeks of recovery (C) the staining shows necrosis in the CA1 sector with neuronal loss and eosinophilic material (arrow).

FIG. 2 depicts 200× sections of hippocampus stained with the βAPP antibody. The results show a weak continuous staining in the cytoplasm and dendrites of neurons in CA1 sector in the sham operated animals (A). In the ischemic animals at four weeks of recovery (B) the staining is observed as granules of βAPP in strata radiatum and oriens. Astrocytes might also be stained with βAPP antibody (arrows). At ten weeks of recovery (C) the punctate pattern in strata radiatum and oriens can still be observed but the immunoreactivity is more obvious in the pyramidal layers as deposits of βAPP (arrow) and neuronal remnants are diffusely stained.

FIG. 3 depicts strata radiatum and oriens CA1. Immunostaining with anti-Aβ-peptide antibody reveals a punctate pattern in strata radiatum and oriens CA1. At four weeks of recovery (A) the immunoreactivity is observed as small accumulation of Aβ. Some cellbodies that appear to be astrocytes are diffusely stained and revealed a punctate pattern (arrows). There is accumulation of Aβ around blood vessels (arrowhead). At ten weeks post ischemia (B) the immunostaining is less intensive and Aβ appears to be degraded. The magnification of the section shown is 200×.

FIG. 4 depicts a 200× section of hippocampus that was stained with anti-α-secretase antibody. β-secretase immunoreactivity in hippocampus at four weeks post ischemia (A) shows an intensive punctate pattern in the pyramidal layer. Some cellboides that appear to be astrocytes show diffuse immunostaining (arrows). At eight weeks post ischemia (B) β-secretase appears to be accumulated in some areas of the pyramidal layer (arrows).

FIG. 5 depicts a 200× section of the parietal cortex stained with βAPP antibody. In the parietal cortex, immunostaining with the βAPP antibody in sham operated animals shows staining the cytoplasm of neurons and in the dendrites (A). At four weeks after ischemia (B) the immunostaining of βAPP is observed in the cytoplasm of neurons and in several dendrites. At ten weeks (C) βAPP is accumulated in the cytoplasm and some neurons show staining in the whole cell (arrowhead). Extracellular deposits of βAPP can also be observed (arrow).

FIG. 6 depicts a 200× section of the parietal cortex stained with anti-Aβ-peptide antibody. In the parietal cortex at four weeks of recovery, immunostaining with anti-Aβ-peptide antibody shows a punctate pattern in the plasma membrane of many neurons.

FIG. 7 depicts a 200× section of the parietal cortex stained with anti-β-secretase antibody. Immunostaining with this antibody in the parietal cortex at four weeks of recovery is observed in the cytoplasm and the whole cell of many neurons. Dendrites also appear to be stained with anti-β-secretase.

FIG. 8 depicts 200× sections of the hippocampus obtained from sham operated rats that were either 3 month (A) or 18 month (B) in age. The hippocampus sections were stained with anti-Aβ antibodies. No anti-Aβ staining is detected in either the young (e.g., about 3 month old) or the aged (e.g., about 18 month old) animal.

FIG. 9 depicts 200× sections of hippocampus that were stained with anti-Aβ antibodies. The anti-Aβ staining reveals much less Aβ deposits accumulated in the young rat (A) at four weeks after the ischemic treatment than that in the similarly treated aged rat (B).

FIG. 10 depicts the same sections of FIG. 9 at a higher magnification.

FIG. 11 depicts 200× sections of hippocampusthat were stained with anti-Aβ antibodies. The anti-Aβ staining reveals a substantial amount of Aβ plaques formed in the aged rat (B) as compared to the similarly treated young rat (A) at 10 weeks after the ischemic treatment.

FIG. 12 depicts the same sections of FIG. 11 at a higher magnification.

FIG. 13 depicts 50× sections of the hippocampus obtained from sham operated rats that were either 3 month (A) or 18 month (B) in age. The hippocampus sections were stained with anti-PAPP antibodies. No anti-βAPP staining is detected in either the young (e.g., about 3 month old) or the aged (e.g., about 18 month old) animal.

FIG. 14 depicts 200× sections of hippocampus obtained from the young (A) or aged rat (B) at four weeks of recovery from the ischemic treatment. The sections were stained with anti-βAPP antibodies.

FIG. 15 depicts 200× sections of hippocampus obtained from the young (A) or aged rat (B) at ten weeks of recovery from the ischemic treatment. The sections were stained with anti-βAPP antibodies.

FIG. 16 depicts 200× sections of hippocampus obtained from the young (A) or aged rat (B) at four weeks of recovery from the ischemic treatment. The sections were stained with anti-β secretase antibodies. The results indicate that β secretase is expressed in the young and the aged animals at four weeks of recovery from the ischemic treatment.

FIG. 17 depicts 200× sections of hippocampus obtained from the aged rat at ten weeks of recovery from the ischemic treatment. The sections were stained with anti-β secretase antibodies. The anti-β secretase staining shows that β secretase continues to be expressed at ten weeks of recovery.

FIG. 18 depicts 50× sections of the hippocampus obtained from sham operated rats that were either 3 month (A) or 18 month (B) in age. The hippocampus sections were stained with anti-β secretase. No anti-β secretase staining is detected in either the young (e.g., about 3 month old) or the aged (e.g., about 18 month old) animal.

### MODE(S) FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains

General Techniques:

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Definitions:

As used in the specification and claims,. the singular form “a”, “an” and “the” include plural references unless the context clearly dictates otherwise. For example, the term “a cell” includes a plurality of cells, including mixtures thereof.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “nucleotide probe” or “probe” refers to a polynucleotide used for detecting or identifying its corresponding target polynucleotide in a hybridization reaction.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR, or the enzymatic cleavage of a polynucleotide by a ribozyme.

A “gene” refers to a polynucleotide containing at least one open reading frame that is capable of encoding a particular protein after being transcribed and translated.

The term “isolated,” as used herein, means separated from other constituents, cellular and otherwise, that in nature is normally associated with the polynucleotide, peptide, polypeptide, protein, antibody, or fragments thereof. As is apparent to those of skill in the art, a non-naturally occurring the polynucleotide, peptide, polypeptide, protein, antibody, or fragments thereof, does not require “isolation” to distinguish it from its naturally occurring counterpart. In addition, a “concentrated,” “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below, under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

A “disease-associated” gene or polynucleotide refers to any gene or polynucleotide which is differentially expressed in a disease condition relative to a non disease control. The “disease-associated” gene may yield a mRNA transcript or translation product at an abnormal level or in an abnormal form in cells derived from disease-affected tissues compared with tissues or cells of a non disease control. As such, a gene associated with a neurodegenerative disorder (e.g. Alzheimer's disease) may be a gene that becomes expressed at an abnormally high level. It also may be a gene that becomes expressed at an abnormally low level, where the altered expression correlates with the occurrence and/or progression of the disease. A disease-associated gene also refers to a gene possessing one or more mutations or a genetic variation that is directly responsible or is in linkage disequilibrium with one or more genes that are responsible for the etiology of a disease. The transcribed or translated products may be known or unknown, and may be at a normal or abnormal level. A disease-associated polypeptide (e.g. AD-associated polypeptide) is the protein product encoded by a disease-associated gene.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in an eukaryotic cell.

“Differentially expressed” or “differential expression” as applied to nucleotide sequence or polypeptide sequence in a subject, refers to over-expression or under-expression of that sequence when compared to that detected in a control. Underexpression also encompasses absence of expression of a particular sequence as evidenced by the absence of detectable expression in a test subject when compared to a control. Differentially expressed or differential expression also encompass an alteration in expression pattern such as tissue distribution, or accumulation of a gene product (e.g., accumulation of Aβ peptide in the brain).

Different polynucleotides are said to “correspond” to each other if one is ultimately derived from another. For example, a sense strand corresponds to the anti-sense strand of the same double-stranded sequence. mRNA (also known as gene transcript) corresponds to the gene from which it is transcribed. cDNA corresponds to the RNA from which it has been produced, such as by a reverse transcription reaction, or by chemical synthesis of a DNA based upon knowledge of the RNA sequence. cDNA also corresponds to the gene that encodes the RNA. A polynucleotide may be said to correspond to a target polynucleotide even when it contains a contiguous portion of the sequence that share substantial sequence homology with the target sequence when optimally aligned.

“Signal transduction” is a process during which stimulatory or inhibitory signals are transmitted into and within a cell to elicit an intracellular response.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

“Mononuclear phagocyte,” as used herein, refers to a target cell of a plaque component and contains specific binding sites required for activation and induction of neurotoxicity. Mononuclear phagocyte is an immune cell, having a single nucleus and the ability to engulf particles, also known as phagocytosis. These cells are found in blood and body tissues including the central nervous system, including the brain. They include microglial cells, monocytes, macrophages, precursor cells of microglia, monocytes, and macrophages, microglia-like cell lines, macrophage-like cell lines, or cell lines modified to express microglia-like surface molecules that are active in accordance with the below definition.

Mononuclear phagocytes may be “activated” by a plaque component following complex formation. Activation is also referred to herein as immune activation, markers of which are any process that renders a mononuclear phagocyte more dynamic characterized by activities such as and not limited to increased movement, phagocytosis, alterations in morphology, and the biosynthesis, expression, production, or secretion of molecules, such as protein, associated with membranes including complement, scavengers, Aβ and blood cell antigens, histocompatibility antigens for example. Production of molecules includes enzymes involved in the biosynthesis of bioactive agents such as nitric oxide synthetase, superoxide dismutase, small molecules such as eicosanoids, cytokines, free radicals and nitric oxide. Release of factors includes proteases, apolipoproteins such as apolipoprotein E, and cytokines such as interleukin-1, tumor necrosis factor as well as other molecules such as hydrogen peroxide.

“Neurotoxins” are defined herein as molecules that injure, damage, kill, or destroy a neuron while sparing other nervous system cells such as glia, for example.

An “animal model” or a “test animal model” refers to a non-human vertebrate, including but not limited to mammal, primate, and rodent. Non-limiting preferred model organisms are rats, mice, guinea pigs, dogs, cats, rabbits, pigs, chimpanzees, and monkeys.

A “control” is an alternative subject or sample used in an experiment for comparison purpose. For example, where the purpose of the experiment is to test whether a candidate modulator is capable ameliorating or advancing a pathogenic condition of AD in a test animal model, it is generally preferable to use a control (an animal or a tissue or cell sample from the animal), which typically exhibits the same or similar pathological condition but is not exposed to the candidate modulator or exposed to a lower dose of the modulator, or for a shorter period of time.

“AD-affected tissues” refer to bodily tissues, especially the brain tissues, which are affected by any one of the pathogenesis steps of AD. As noted above, AD is a multi-step process, involving elevated Aβ peptide production and deposition, plaque formation, mononuclear phagocyte activation, neurofibrillary tangles formation and/or finally neuronal loss. An AD-affected tissue can be derived from artificial plaque models, such as animal models that mimic one or more steps of AD pathogenesis.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975).

By “a therapeutically effective” amount of a drug or pharmacologically active agent or pharmaceutical formulation is meant a nontoxic but sufficient amount of the drug, agent or formulation to provide the desired effect, i.e., inhibiting, preventing, or reversing the onset or progressive course of a neurodegenerative disorder.

As used herein, the term “antibody” refers to a polypeptide or group of polypeptides which are comprised of at least one antibody combining site. An “antibody combining site” or “binding domain” is formed from the folding of variable domains of an antibody molecule(s) to form three-dimensional binding spaces with an internal surface shape and charge distribution complementary to the features of an epitope of an antigen, which allows an immunological reaction with the antigen. An antibody combining site may be formed from a heavy and/or a light chain domain (VH and VL, respectively), which form hypervariable loops which contribute to antigen binding. The term “antibody” includes, for example, vertebrate antibodies, hybrid antibodies, chimeric antibodies, altered antibodies, univalent antibodies, the Fab proteins, and single domain antibodies.

The term “monoclonal antibody” refers to an antibody composition having a substantially homogeneous antibody population. It is not intended to be limited as regards to the source of the antibody or the manner in which it is made. Monoclonal antibodies are highly specific, being directed against a single antigenic site. In contrast to conventional (polyclonal) antibody preparations which typically include different antibodies directed against different determinants (epitopes), each monoclonal antibody is directed against a single determinant on the antigen.

As used herein, a “modulator” encompasses biological or chemical molecules that (a) bind to or interact with AD-associated genes or proteins; (b) molecules that inhibit or activate the AD-associated protein directly or indirectly; (c) molecules that interfere with the interaction between the AD- associated proteins and their upstream or downstream signaling molecules; and, (d) molecules which modulate the AD-associated gene or polypeptide expression profile.

Generation of Animal Models of the Present Invention:

A central aspect of the present invention is the design of an animal model of AD. Accordingly, the present invention provides a method of generating an animal model exhibiting a pathological condition of AD. The method comprises the steps of: (a) inducing a transient forebrain ischemia in the animal; and (b) allowing the animal to recover from the ischemic induction for a sufficient amount of time so that a pathological condition of AD is exhibited.

Induction of Forebrain Ischemia:

The animal models of the present invention encompass any non-human vertebrates that are amenable to procedures yielding an ischemic condition in the animal's brain. Preferred model organisms include but are not limited to mammals, primates, and rodents. Non-limiting examples of the preferred models are rats, mice, guinea pigs, dogs, cats, rabbits, pigs, chimpanzees, and monkeys.

As used herein, ischemic condition in the forebrain generally refers to a transient reduction in cerebral blood flow. Such forebrain ischemia can be achieved using a number of well-established procedures in the art (see, e.g., Pulsinell et al. (1979) Stroke 10:267-272). A preferred method is described in detail in Smith et al. (1984) Acta Neuro Scand 69:385-401. This method allows a rapid reduction of blood pressure to about 50 mm Hg during a period of approximately 10 minutes. In this method, ischemia is induced by a combination of bilateral carotid artery clamping and reduction of mean arterial blood pressure, the latter being achieved by bleeding either with or without pharmacological support. To reduce blood pressure, a catheter may be inserted in an external jugular vein. Alternatively, blood can be drawn from tail artery catheter after vasodilation with either i.v. Arfonad® or Regitin®. At the same time, the carotid arteries are occluded and the blood is maintained at about 50 mm Hg. The ischemic condition is typically maintained for about 10 minutes, preferably for about 15 to about 20 minutes. Ischemia is terminated by releasing carotid artery clamping and by reinfusion of the shed blood.

After induction of a transient forebrain ischemia, the animal is allowed to recover for a sufficient amount of time to effect the development and exhibition of a pathological condition characteristic of AD. While the amount of time required for developing Alzheimer-like pathology varies among different animals, it generally requires at least about 2 weeks, more often requires at least about 4 weeks, and even more often requires about 4 to about 10 weeks. During recovery, the animals are typically kept at room temperature and in cages with access to water and food.

Detection of exhibited pathological condition of Alzheimer's disease:

Following an initial phase of degeneration of brain tissues within minutes to hours from the transient ischemic insult, a delayed phase of pathological condition characteristic of AD occurs in about 2 to 10 weeks, more often about 4 to 10 weeks in the animal models of the present invention. The terms “pathological condition of AD,” “AD pathological condition,” “pathological condition characteristic of AD,” or “AD-like pathological condition” are used herein interchangeably. They refer to a pathogenic state of the central nervous system that is characteristic of AD. In one aspect, the pathological condition is characterized by a differential expression of an AD-associated gene or polypeptide. Preferred AD-associated genes or polypeptides are those whose overexpression or underexpression in the central nervous system are indicative of AD. An illustrative example of this category of AD-associated polypeptide is Aβ peptide. The accumulation of Aβ peptide is a defining feature of AD. Other preferred AD-associated polypeptides include but: are not limited to beta-secretase, tau, hyperphosphorylated tau, ApoE, ubiquitin and retinol binding protein. In another aspect, contemplated Alzheimer-like pathological conditions are plaque formation, mononuclear phagocyte activation, neurofibrillary tangles formation and/or neuronal loss.

The differential expression of the AD-associated gene or polypeptide is determined by assaying for a difference, between a test biological sample (derived from the animal model of the invention) and a control sample, in the level of polypeptides or the corresponding transcripts.

The test sample used for this invention can be brain tissues including solid hippocampal tissues or cortex tissue, tissue cultures or cells derived therefrom and the progeny thereof, and sections or smears prepared from the source, or any other samples of the brain that contain nucleic acids. The selection of an appropriate control sample is dependent on the test sample cell or tissue initially selected and its phenotype which is under investigation. Whereas the test sample is derived from an AD animal model of the present invention, one or more counterpart can be used as a control. Counterparts can include, for example, normal brain tissues that have not been subjected to an ischemic insult (e.g., sham animals). Additionally, the counterparts can be normal brain tissues that lack Aβ complex plaques, or normal cell lines that are established from the normal brain tissues. Preferably, a control matches the tissue, and/or cell type the tested sample is derived from. It is also preferable to analyze the control and the tested sample in parallel.

As is known to those of ordinary skill in the art, any technique capable of measuring protein contents is generally useful for determining a differential expression of the AD-associated polypeptides. Determining the protein level typically involves (a) contacting the polypeptides contained in the biological sample with an agent that specifically binds a polypeptide encoded by the AD-associated genes; and (b) identifying any agent:polypeptide complex so formed. In one aspect of this embodiment, the agent that specifically binds an AD-associated polypeptide is an antibody, preferably a monoclonal antibody.

The reaction is performed by contacting the agent with a sample of polypeptides derived from the test sample under conditions that will allow a complex to form between the agent and AD-associated polypeptide. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is more important, however, that the label does not interfere with the binding reaction. In the alternative, an indirect detection procedure requires the agent to contain a label introduced either chemically or enzymatically, that can be detected by affinity cytochemistry. A desirable label generally does not interfere with binding or the stability of the resulting agent:polypeptide complex. However, the label is typically designed to be accessible to an antibody for an effective binding and hence generating a detectable signal. A wide variety of labels are known in the art. Non-limiting examples of the types of labels that can be used in the present invention include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the AD-associated polypeptide is tested for its ability to compete with a labeled analog for binding sites on the specific agent. In this competitive assay, the amount of label captured is inversely proportional to the amount of AD-associated polypeptide present in a test sample.

A variety of techniques for protein analysis using the basic principles outlined above are available in the art. They include various formats of immunoassays such as radioimmunoassays, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and any proteomic analytic tools. In addition, cell sorting analysis can be employed to detect cell surface antigens. Such analysis involves labeling target cells with antibodies coupled to a detectable agent, and then separating the labeled cells from the unlabeled ones in a cell sorter. A sophisticated cell separation method is fluorescence-activated cell sorting (FACS). Cells traveling in single file in a fine stream are passed through a laser beam, and the fluorescence of each cell bound by the fluorescently labeled antibodies is then measured.

Antibodies that specifically recognize and bind to the protein products of interest are required for conducting the aforementioned protein analyses. These antibodies may be purchased from commercial vendors or generated and screened using methods well-established in the art.

The differential expression of the AD-associated polypeptide can also be determined by assaying for a difference, between the test biological sample and the control sample, in the level of transcripts encoding the differentially expressed polypeptide.

Such a comparative gene expression analysis on the polynucleotides obtained from the test sample and the control sample can be performed by hybridization techniques well established in the art. Representative procedures include but are not limited to cDNA subtraction, differential display (Liang et al. (1992) Science 257:967-971), Serial Analysis of Gene Expression or “SAGE” (Velculescu, et al. (1995) Science 270:484-487 and U.S. Pat. No. 5,695,937), and array-based methodology (see, e.g., U.S. Pat. No. 5,445,934).

The recently emerged array-based analysis is particularly preferred for comparative gene expression profiling. The array-based technology involves hybridization of a pool of target polynucleotides corresponding to gene transcripts of a test sample to an array of tens and thousands of probe sequences immobilized on the array substrate. The technique allows simultaneous detection of multiple gene transcripts and yields quantitative information on the relative abundance of each gene transcript expressed in a test subject. By comparing the hybridization patterns generated by hybridizing different pools of target polynucleotides to the arrays, one can readily obtain the relative transcript abundance in two pools of target samples. The array analysis can be extended here to detecting differential expression of genes between AD-affected and normal tissues, among different types of AD-affected tissues and cells, amongst cells at different disease stages, and amongst cells that are subjected to various candidate modulators of AD-associated genes or proteins.

In assaying for an alteration in the level of mRNA transcripts or corresponding polynucleotides, nucleic acid contained in the aforementioned samples is first extracted according to standard methods in the art. For instance, mRNA can be isolated using various lytic enzymes or chemical solutions according to the procedures set forth in Sambrook et al. (1989), supra or extracted by nucleic-acid-binding resins following the accompanying instructions provided by manufactures. The mRNA contained in the extracted nucleic acid sample is then detected by hybridization (e.g. Northern blot analysis) and/or amplification procedures according to methods widely known in the art or based on the methods exemplified herein.

Nucleic acid molecules having at least 25 nucleotides and exhibiting sequence complementarity or homology to the polynucleotides encoding the AD-associated polypeptides find utility as hybridization probes. It is known in the art that a “perfectly matched” probe is not needed for a specific hybridization. Hybridization can be performed under conditions of different “stringency.” Relevant conditions include temperature, ionic strength, time of incubation, the presence of additional solutes in the reaction mixture such as formamide, and the washing procedure. Higher stringency conditions are those conditions, such as higher temperature and lower sodium ion concentration, which require higher minimum complementarity between hybridizing elements for a stable hybridization complex to form. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC.

Polynucleotide sequences that hybridize under conditions of greater stringency are more preferred. As is apparent to one skilled in the art, hybridization reactions can accommodate insertions, deletions, and substitutions in the nucleotide sequence. Thus, linear sequences of nucleotides can be essentially identical even if some of the nucleotide residues do not precisely correspond or align. The relationship between hybridization stringency, degree of sequence identity, and polynucleotide length is known in the art and can be calculated by standard formulae.

In assaying for the presence of differential expression of AD-associated genes, probes are allowed to form stable complexes with the target polynucleotides contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Suitable hybridization conditions for the practice of the present invention are such that the recognition interaction between the probe and target is both sufficiently specific and sufficiently stable. As noted above, hybridization reactions can be performed under conditions of different “stringency”.

Conditions that increase the stringency of a hybridization reaction are widely known and published in the art. See, for example, (Sambrook, et al., (1989), supra; Nonradioative In Situ Hybridization Application Manual, Boehringer Mannheim, second edition). The hybridization assay can be formed using probes immobilized on any solid support, including but are not limited to nitrocellulose, glass, silicon and metal. A preferred hybridization assay is conducted on high-density arrays as described in the above section (see also U.S. Pat. No. 5,445,934).

For a convenient detection of the probe-target complexes formed during the hybridization assay, the nucleotide probes are conjugated to a detectable label. Detectable labels suitable for use in the present invention include any composition detectable by spectroscopic, photochemical, biochemical, immunochemical, electrical, optical or chemical means. A wide variety of appropriate detectable labels are known in the art, which include luminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to determine where hybridization has taken place and/or to quantify the hybridization intensity will typically depend upon the label selected above. For example, radiolabels may be detected using photographic film or a phosphoimager. Fluorescent markers may be detected and quantified using a photodetector to detect emitted light (see U.S. Pat. No. 5,143,854 for an exemplary apparatus). Enzymatic labels are typically detected by providing the enzyme with a substrate and measuring the reaction product produced by the action of the enzyme on the substrate; and finally colorimetric labels are detected by simply visualizing the colored label.

One of skill in the art, however, will appreciate that hybridization signals will vary in strength with efficiency of hybridization, the amount of label on the target nucleic acid and the amount of particular target nucleic acid in the sample. In evaluating the hybridization data, a threshold intensity value may be selected below which a signal is not counted as being essentially indistinguishable from background. In addition, the provision of appropriate controls permits a more detailed analysis that controls for variations in hybridization conditions, non-specific binding and the like. Where desired, a normal or standard expression profile of a given AD-associated gene can be established for a comparative analysis by, e.g., using reliable data generated from replicate spots, replicated biological specimens for probes and statistical analysis of comparisons of experimental and control probes. Typically, statistical tests include Student's t-test, ANOVA analysis and/or pattern recognition methods.

Uses of the Animal Models of the Present Invention:

The subject animal models provide an effective tool to elucidate the underlying molecular basis of AD, and to develop diagnostics and therapeutics for AD. The animal models have a wide variety of utilities including (a) identification and quantification of differential gene expression between diseased and normal tissues; (b) the detection of perturbations or abnormalities in the signal transduction pathway mediated by AD-associated genes or proteins; (c) the screen of modulators of AD pathogenesis; and (d) the validation of modulators that are capable of ameliorating a pathological condition of AD.

Accordingly, the present invention provides a method of developing a modulator of pathogenesis of AD, comprising: (a) administering a candidate modulator to a test animal model generated by a method comprising (i) inducing a transient and reversible forebrain ischemia in the animal; and (ii) allowing the animal to recover from the ischemic induction for a sufficient amount of time so that a pathological condition of AD is exhibited; and (b) detecting a change in the pathological condition in the test animal model of (a) relative to a control.

Where desired, the control can be an animal to which the candidate modulator is not administered or is administered at a lower dose or for a shorter period of time relative to the test animal. The control can be an animal to which a known modulator whose effect has been previously characterized is administered for a comparison. Preferably, the control animal exhibits the same or similar pathological condition. The control animal may also be generated by the same method used to generate the test animal.

A change in the activity or expression level is indicative of a candidate therapeutic modulator. If the modulator is neuroprotective, it when administered into a cell or subject may reduce the level of expression or activity of an AD-causing gene or protein. Alternatively, it may augment the level of expression or activity of an AD-suppressing gene or protein.

A modulator-induced change in the AD-associated protein expression can be assayed by any conventional techniques known in the art. All of the aforementioned gene expression analyses are in general applicable for practicing this embodiment.

The assay for a modulator-induced change in the activity of an AD-associated protein is generally dependent on the nature of the AD-associated protein and its signal transduction pathway that is under investigation. For example, where the AD-associated protein is part of a signaling cascade involving a fluctuation of intracellular pH condition, pH sensitive molecules such as fluorescent pH dyes can be used as the reporter molecules. In another example where the AD-associated protein is a kinase, the ability of the protein to conduct autophosphorylation or phosphorylation of an appropriate target can be measured by a variety of kinase assays available in the art. Where the AD-associated protein is a phosphatase, its ability to dephosphorylate an appropriate substrate can also be conveniently detected. In yet another example, where the AD-associated protein is proteinase, its ability to cleave a substrate can also be detected by measuring the release of the cleaved polypeptide or the reduction of the substrate. In yet another example where the AD-associated protein is an ion channel, fluctuations in membrane potential and/or intracellular ion concentration can be monitored.

A number of high-throughput assays and devices are particularly suited for a rapid and robust screening for these and other classes of modulators. Representative assays are beta-lactamase assay (Aurora Biosciences) and AlphaScreen™ Amplified Luminescent Promximity Homogenous Assay (Perkin Elmer). Illustrative high throughput instruments include FLIPR™ (Molecular Devices, Inc.) and VIPR (Aurora Biosciences). These instruments are capable of performing stimulation in over 100 wells of samples contained in a microplate simultaneously, and providing real-time measurement and functional data once every second. Typically, the assay is completed in less than fifteen minutes. Since more than hundred microplates can be read in a day, nearly 10,000 different candidate AD modulators can be tested.

Of particular interest are modulators that are capable of ameliorating a pathological condition of AD. Preferably, the modulators are capable of reducing or inhibiting the accumulation of beta-amyloid peptide, accumulation and/or activation of beta-secretase, tau, hyperphosphorylated tau, ApoE, ubiquitin and retinol binding protein. More preferably, the modulators are capable of ameliorating other contemplated Alzheimer-like pathological conditions, such as plaque formation, mononuclear phagocyte activation, neurofibrillary tangles formation and/or neuronal loss. The modulators may act at any point of the signal transduction pathway leading to one or more of the aforementioned pathological conditions. A summary of the signaling cascades underlying the pathogenesis of AD is found in U.S. Pat. No. 6,071,493, which is incorporated herein by reference.

Another class of modulators of particular interest includes modules capable of advancing a pathological condition of AD. These modules may mediate such adverse effect by reducing the level of expression or activity of an AD-suppressing gene or protein. Alternatively, it may augment the level of expression or activity of an AD-causing gene or protein. These modulators may also act at any point of the signal transduction pathway leading to one or more of the aforementioned pathological conditions. Identification of this class of modulators provides additional insights into the structures and mechanisms of molecules that accelerate AD pathogenesis, and thus facilitating a rapid design and development of agents which prevent or counter the adverse effect.

Candidate modulators of the present invention include a biological or chemical compound such as a simple or complex organic or inorganic molecule. Such compounds may include, but are not limited to, antisense oligonucleotide, small interfering RNA, a ribozyme, a ribozyme derivative, liposomes, peptides such as, for example, soluble peptides, including but not limited to members of random peptide libraries; (see, e.g., Lam, K. S. et al., 1991, Nature 354:82-84; Houghten, R. et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang, Z. et al., 1993, Cell 72:767-778); molecules from natural product libraries, antibodies (including, but not limited to, polyclonal, monoclonal, humanized, anti-idiotypic, chimeric or single chain antibodies, and FAb, F(ab′)₂ and FAb expression library fragments, and epitope-binding fragments thereof). In addition, a vast array of small organic or inorganic compounds from natural sources such as fungal, plant or animal extracts, and the like, can be employed in the screening assay. It should be understood, although not always explicitly stated, that the modulator is used alone or in combination with another modulator, having the same or different biological activity as the modulators identified by the inventive screen. The identified modulators are particularly useful in AD therapies.

In practicing this embodiment, candidate modulators can be administered into the animal model through a wide variety of routes of administration and dosage forms. Any suitable route of administration may be employed for providing a mammal, primate, rodent, and especially a rat, an effective dosage of a candidate modulator of the present invention. For instance, the candidate modulators can be administered intravenously, subcutaneously, intramuscularly, intraperitoneally, intradermally, orally, intranasally, or intrapulmonarily. Illustrative dosage forms include tablets, troches, dispersions, suspensions, solutions, capsules, creams, ointments, aerosols, and the like. The effective dosage of active ingredient employed may vary depending on the particular modulators employed, the mode of administration, the condition being treated and the severity of the condition being treated. Such dosage may be ascertained readily by a person skilled in the art.

The present invention provides an alternative method for developing a modulator of an AD-associated gene or protein. The method comprises: (a) contacting a candidate modulator with an AD-associated gene or protein that is contained in a test biological sample derived from an animal model, wherein the animal model is generated by a method comprising: (i) inducing a transient forebrain ischemia in the animal; and (ii) allowing the animal to recover from the ischemic induction for a sufficient amount of time so that a pathological condition of AD is exhibited; and (b) detecting an alteration in expression of the AD-associated gene or protein, or an alteration in activity of the protein of step (a), relative to a control sample.

In one aspect, the modulator so identified ameliorates a pathological condition of AD. Such pathological condition includes but is not limited to the accumulation of beta-amyloid peptide, accumulation and/or activation of beta-secretase, tau, hyperphosphorylated tau, ApoE, ubiquitin and retinol binding protein. In another aspect, the modulators ameliorate other contemplated Alzheimer-like pathological conditions, such as plaque formation, mononuclear phagocyte activation, neurofibrillary tangles formation and/or neuronal loss. In yet another aspect, the modulator advances a pathological condition of AD, including one or more of the aforementioned conditions.

The practice of this embodiment of the invention involves a comparison of the expression or activity of an AD-associated gene or protein present in the test biological sample relative to a control sample.

The test sample used for this method can be brain tissues of an animal model of the present invention, including solid hippocampal tissues or cortex tissue, tissue cultures or cells derived therefrom and the progeny thereof, and sections or smears prepared from the source, or any other samples of the brain that contain nucleic acids or polypeptides.

The control sample includes counterparts of the test sample used for a purpose of comparison. Such counterparts may be a sample derived from the animal model that is not exposed to the candidate modulator, or is exposed to a lower dose of the modulator for the same or a shorter period of time. The control can be a sample to which a known modulator whose effect has been previously characterized is added for a comparison. Preferably, the control sample is derived from a test animal model exhibiting the same or similar pathological condition. The control animal may also be generated by the same method used to generate the test animal model of the present invention.

To practice the method in vitro, typically cell cultures or tissue cultures are first provided. The cells are cultured under conditions (temperature, growth or culture medium and gas (CO₂)) and for an appropriate amount of time to attain exponential proliferation without density dependent constraints. It also is desirable to maintain an additional separate cell culture; one which does not receive the candidate modulator being tested as a control.

The general parameters governing eukaryotic cell survival are well established in the art. Physicochemical parameters which may be controlled in vitro are, e.g., pH, CO₂, temperature, and osmolarity. The nutritional requirements of cells are usually provided in standard media formulations developed to provide an optimal environment. Nutrients can be divided into several categories: amino acids and their derivatives, carbohydrates, sugars, fatty acids, complex lipids, nucleic acid derivatives and vitamins. Apart from nutrients for maintaining cell metabolism, most cells also require one or more hormones from at least one of the following groups: steroids, prostaglandins, growth factors, pituitary hormones, and peptide hormones to survive or proliferate (Sato, G. H., et al. in “Growth of Cells in Hornonally Defined Media”, Cold Spring Harbor Press, N.Y., 1982; Ham and Wallace (1979) Meth. Enz., 58:44, Barnes and Sato (1980) Anal. Biochem., 102:255, or Mather, J. P. and Roberts, P. E. (1998) “Introduction to Cell and Tissue Culture”, Plenum Press, New York.

Following these general techniques, a variety of cells derived from the brain tissues have been successfully cultured. Representative cell types include a wide range of mononuclear phagocytes and certain neuronal cells.

The subject method involves contacting a candidate modulator with a test cell expressing an AD-associated gene or protein. The candidate modulator may be by directly added to the cell culture or added to culture medium. As is apparent to those skilled in the art, an “effective” amount must be added which can be empirically determined.

Where the candidate modulator is a nucleic acid, it can be added to the cell cultures by methods well known in the art, which includes, but is not limited to calcium phosphate precipitation, microinjection or electroporation. Alternatively or additionally, the nucleic acid can be incorporated into an expression or insertion vector for incorporation into the cells. Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression. Examples of vectors are viruses, such as baculovirus and retrovirus, bacteriophage, adenovirus, adeno-associated virus, cosmid, plasmid, fungal vectors and other recombination vehicles typically used in the art which have been described for expression in a variety of eukaryotic hosts. Among these are several non-viral vectors, including DNA/liposome complexes, and targeted viral protein DNA complexes. To enhance delivery to a cell, the nucleic acid or proteins of this invention can be conjugated to antibodies or binding fragments thereof which bind cell surface antigens. Liposomes that also comprise a targeting antibody or fragment thereof can be used in the methods of this invention.

The comparative analysis on the gene expression or protein activity in response to treatment with a candidate modulator may involve hybridization and/or immunoassays. A variety of hybridization and immunoassays are available in the art. One may choose to employ any techniques described in the above sections or other procedures known in the art. Likewise, any of the aforementioned technique applicable for detecting the activity of a protein can be employed to practice this method.

Pharmaceutical Compositions of the Present Invention:

The present invention provides pharmaceutical compositions containing modulators developed using one or more of the above-described methods. Such pharmaceutical compositions are useful for ameliorating a pathological condition of AD, either alone or in conjunction with other forms of therapy.

The preparation of pharmaceutical compositions of this invention is conducted in accordance with generally accepted procedures for the preparation of pharmaceutical preparations. See, for example, Remington's Pharmaceutical Sciences 18th Edition (1990), E. W. Martin ed., Mack Publishing Co., PA. Depending on the intended use and mode of administration, it may be desirable to process the active ingredient further in the preparation of pharmaceutical compositions. Appropriate processing may include sterilizing, mixing with appropriate non-toxic and non-interfering components, dividing into dose units, and enclosing in a delivery device.

Liquid pharmaceutically acceptable compositions can, for example, be prepared by dissolving or dispersing a modulator embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

Pharmaceutical compositions of the present invention are administered by a mode appropriate for the form of composition. Typical routes include subcutaneous, intramuscular, intraperitoneal, intradermal, oral, intranasal, and intrapulmonary (i.e., by aerosol). Pharmaceutical compositions of this invention for human use are typically administered by a parenteral route, most typically intracutaneous, subcutaneous, or intramuscular.

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or liquid aerosol when used with an appropriate aerosolizer device. Although not required, pharmaceutical compositions are preferably supplied in unit dosage form suitable for administration of a precise amount. Also contemplated by this invention are slow release or sustained release forms, whereby a relatively consistent level of the active compound are provided over an extended period.

The invention may be better understood by reference to the following examples, which are intended to merely illustrate but not limit the mode now known for practicing the invention.

### EXAMPLES

### Example 1

### Generation of Animal Models of the Present Invention

Materials and Methods:

All animal experiments were approved by ethical committee at Lund University. Male Wistar rats weighing 325-350 g (M&B A/S Ry, Denmark) were used in the following studies. The forebrain cerebral ischemia was induced by a two-vessel occlusion procedure similar to that described by Smith and co-workers 1984 (Smith, et al. (1984a) Acta Neurol Scand 69:385-401).

Prior to the surgery the animals were fasted over night with free access to water. All experimental rats were deeply anesthetized with 3.5% fluothane (Astra lakemedel, Sweden) in oxygen/nitrous oxide (30/70%). Following intubation, the rats were artificially ventilated, and the flouthane concentration was reduced to 1.5%. A tail artery was cannulated for blood pressure recording and for measurement of blood gases. A tail vein was cannulated for infusion of a muscle relaxant (norcuron/vercuronium bromide, (Organon, Baxtel, Holland), during the ischemic period. A soft catheter was placed into the inferior caval vein via the jugular vein to control the MABP (mean arterial blood pressure) during the ischemic period. Both common carotid arteries were isolated and encircled by loose ligatures for a later transient occlusion. For registration of electro-encephalogram (EEG) two bipolar EEG electrodes were placed in the temporal muscles, and a reference electrode were placed in the femoralis muscle. The body and skull temperatures were monitored before, during and following ischemia (15 minutes of reperfusion) using temperature probes in rectum and subcutaneously under the scalp. Heating pad and a lamp were used to keep the temperature close to 37° C. At the end of the surgery, the flouthane was decreased to 0.5% and continuous i.v. infusion with 2 mg/h norcuron was started. The rats were subjected to a steady state period of 20-30 minutes and blood gases were measured at least two times to keep pCO₂ between 35-40 mmHg and PO₂ around 100 mmHg. Prior to first the measurement 0.2 ml heparin (300 IU/ml) was given in the jugular vein catheter.

Induction of Ischemia:

Immediately prior to ischemia a bolus dose of 0.2 ml norcuron (2 mg/ml) was given in the jugular vein. To induce ischemia blood was withdrawn via the jugular vein catheter for a rapid reduction in blood pressure to 50 mmHg combined with bilateral occlusion of the carotid arteries by surgical clips. The MABP was kept at 50 mmHg during ten minutes (the ischemic period) and EEG was controlled to be isoelectric. Reperfusion was started with re-infusion of blood through the jugular vein. At a blood pressure of around 80 mmHg the occlusion of carotid arteries was released. A dose of 0.5 ml of 0.6 M sodium bicarbonate was administered with rest of the blood to avoid acidosis. At ten minutes of recirculation the blood gases were controlled to be within normal ranges. The animals were allowed to wake up, and when spontaneous breathing recurred they were extubated and transferred to cages with food and water ad libitum.

Sham operated animals were treated in the same way as the ischemic rats, but without occlusion of carotid arteries and reduction in blood pressure.

### Example 2

### Detection of a Pathological Condition in the Animal Models of the Present Invention

Tissue Preparation and Immunohistochemistry:

The ischemic animals were sacrificed at four (n=4), five (n=4), eight (n=5) and ten (n=4) weeks after the ischemic insult. The sham operated animals were sacrificed at four (n=2), five (n=2), eight and ten (n=3) weeks post operation. Under deep flouthane anesthesia, the rats were perfused by intra aortic infusion of 4% paraformaldehyd in 0.1 M phosphate buffer pH 7.4, after a brief washout of the vasculature with 0.9% sodium chloride. The brains were then removed and placed in fixative for 1 h and then in 70% ethanol or in 25% sucrose in 0.1M phosphate buffer. The brains in 70% ethanol were dehydrated, embedded in paraffin and cut coronally in 5 μm thick sections. The brains in 25% sucrose were sectioned on dry ice into 30 μm thick sections which were stored in anti-freeze medium (30% ethylenglycol, 30% glycerol in 0.5 M phosphate buffer) at −20° C. for later immunostaining procedures.

To control the morphology of the sections all brains were stained with celestine blue and acid fuchsin described by Auer and co-workers 1984 with the modification that celestine blue was used as background staining instead of cresyl violet. Auer, et al. (1984) Diabetes 33:1090-1098.

To the immunostaining of the βAPP a polyclonal antibody to the N-terminal region of the amyloid precursor protein (βAPP 1:200; Sigma-Aldrich) was used. Immunostaining of the Aβ was performed with a polyclonal antibody to the human beta-amyloid peptide 1-42 (Aβ 1:100; Alpha Diagnostic International (ADI)). In the immunostaining of the β-secretase a polyclonal antibody to BACE (1:20; Affinity bioreagents—Scandinavian diagnostic services (SDS)) was used. All of the antibodies were tested on both paraffin sections and free floating sections. Secondary antibodies used in the study were biotinylated swine anti rabbit (1:400; Dako AB), biotinylated horse anti mouse (1:200; Vektor Lab.—Immunkemi) and CY2 conjugated streptavidin (1:400; Jackson Immuno Research). The serum used were normal swine serum (Dako AB) and normal horse serum (Vektor Lab.—Immunkemi).

To test for non-specific staining, negative controls were used. In the staining with βAPP the primary antibody was omitted and instead rabbit IgG (1:200(0.05 μg/μl); Sigma-Aldrich) was used. Then the sections were processed in the same way as sections with primary antibody. In the staining with Aβ and β-secretase we used control peptides (used for the generation of the primary antibody). According to the protocols from the company the primary antibodies were allowed to react with their respective peptide in a test tube. After a specific time the solutions were reacted with the sections in the same way as the primary antibodies and were then treated as the rest of the sections.

Before staining, paraffin sections were deparaffinated in xylene and then rehydrated in 99.5%, 95% and 70% ethanol. The sections were then treated with 99% formic acid for 5 minutes, Vinters, et al. (1988) Am J Pathol 133:150-162, followed by tap water rinse for ten minutes. Then the sections were placed in 0.3% H₂O₂ in KPBS (potassium phosphate buffered saline), pH 7.4 to block endogenous peroxidase activity and were then washed in KPBS 2×15 minutes. Each section was encircled by a water-resistant pen (Vector Lab.—Immunkemi) and incubated with 5% normal serum (from the same species in which the secondary antibody was made) in KPBS with 0.25% tritonX-100 (KPBS/TX) for 1 hour at room temperature. The step was followed by incubation with the primary antibody diluted in 5% normal serum in KPBS/TX over night at 4° C. Next day, after rinsing in KPBS/TX, biotinylated secondary antibody in 2% normal serum in KPBS/TX was applied for one hour at room temperature. The vectastain ABC (avidin biotin complex) method (Vector Lab.—Immunkemi) and 3,3′diaminobenzidine (DAB) (Dako AB) with NiCl₂.6H₂O (70 μl 8% NiCl₂/20 ml DAB solution) were used for visualization of primary antibody binding. After rinsing the ABC complex was reacted with the sections for one hour at room temperature and then the sections were rinsed in KPBS followed by Ni-DAB treatment. The peroxidase reaction was started with 4 μl 3% fresh H₂O₂/ml DAB solution and the reaction was stopped by putting sections in ice-cold KPBS. The sections were then dehydrated in rising ethanol concentration and after clearing in xylene cover slips were mounted with Pertex (Histolab products AB).

Free-floating sections were treated in the same way except for the deparaffination, re-hydration and formic acid treatment steps. The sections were processed through all steps as free-floating sections. After DAB reaction and rinsing in KPBS the sections were mounted on glass and allowed to dry to the glass over night. Next day the sections were dehydrated cleared and mounted with cover slips.

Identification of Immunoreactivity:

Representative areas of hippocampal CA1 sector ˜3.8 mm posterior to bregma, Paxinos et al. (1986), 2^{nd} ed., and neocortex were examined and digitalized on a computer connected to a light or a fluorescence microscope.

Results:

Histology

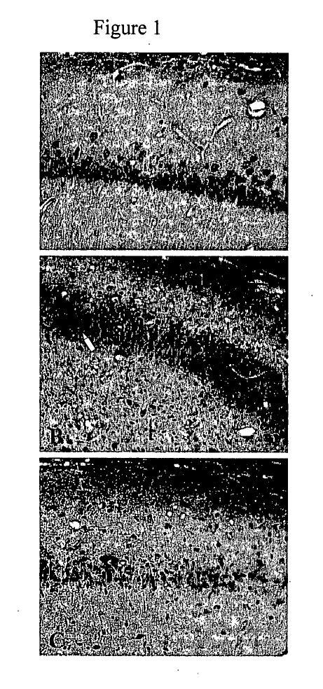

With celestine blue and acid fuchsin staining all of the sham operated animals showed a normal morphology with violet stained neurons with rounded cells and large nuclei (FIG. 1A).

All of the ischemic rats showed necrosis in the CA1 sector with neuronal loss and also with eosinophilic material (FIGS. 1B and C).

Negative Controls:

The negative controls for the βAPP, Aβ, and β-secretase staining were as follows: The rabbit IgG gave some background staining that was distinct from that with the βAPP antibody. The staining with antibody was completely lost when anti-Aβ-antibody was blocked. When the β-secretase antibody was pre-incubated with the corresponding peptide only weak staining remained.

Immunohistochemistry of Hippocampus:

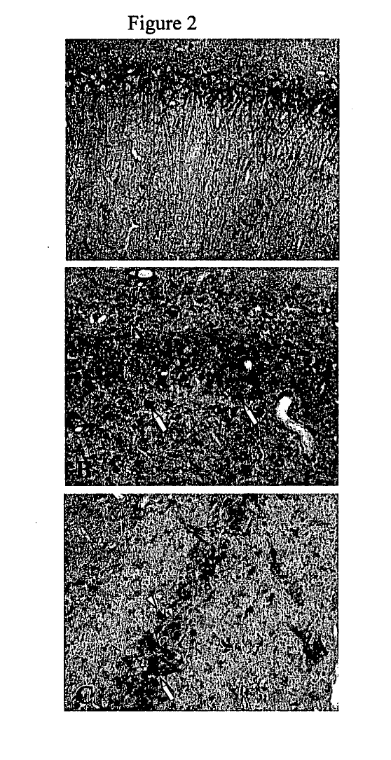

Immunostaining with antibody directed to βAPP (FIG. 2) in sham operated animals showed a continuous staining in the cytoplasm and the dendrites of neurons in the pyramidal cell layer of CA1 (FIG. 2A). In the ischemic animals immunolabeling could be seen as extensive granular βAPP deposition in strata radiatum and strata oriens at four weeks of recovery. Diffuse immunostaining was also seen in some cells that appeared to be astrocytes (FIG. 2B). At ten weeks of recovery there were still granules in strata oriens and radiatum but the staining was more concentrated to the pyramidal layer, as intensively stained deposits of βAPP also neuronal remnants were diffused stained (FIG. 2C).

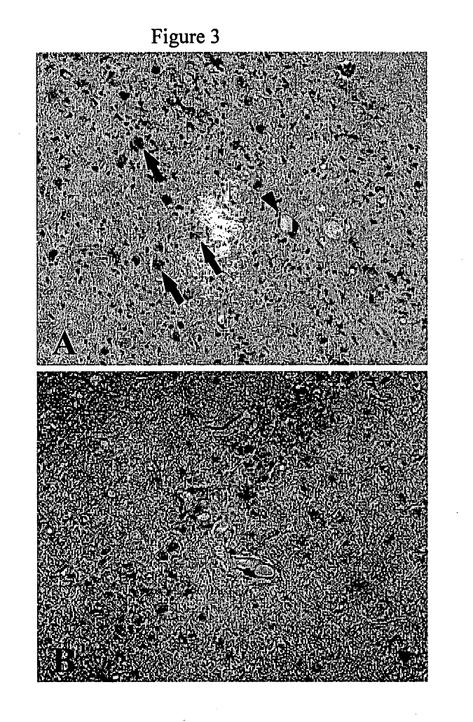

Immunostaining with the Aβ antibody (FIG. 3) revealed a prominent granular staining and smaller accumulations of Aβ in CA1. The staining was spread both in strata radiatum and oriens. Diffuse staining of cells, probably astrocytes, containing stained granules could be seen at four weeks of recovery. Staining around blood vessels could be seen both as punctate pattern and as granular depositions of Aβ (FIG. 3A). At ten weeks of recovery the intense in staining decreased and the amount of Aβ seemed to be reduced (FIG. 3B).

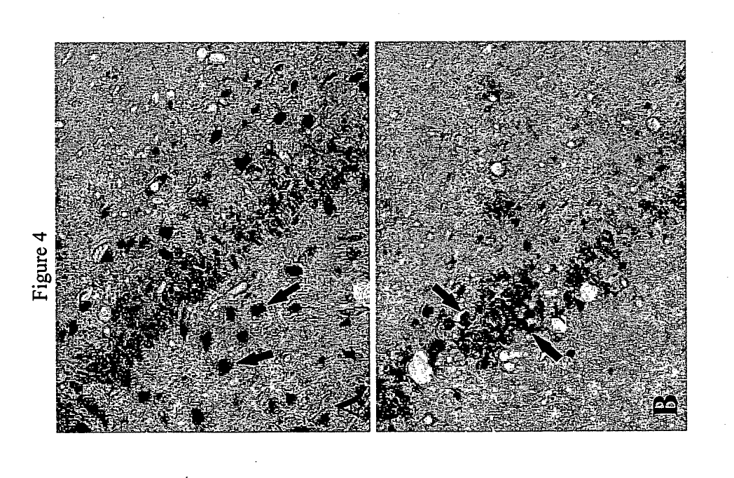

Immunostaining with the antibody to β-secretase (FIG. 4) was seen in the CA1 region as intensive spots in the pyramidal layer at four weeks of survival (FIG. 4A). Some cells that appeared to be astrocytes showed diffuse immunolabelling. At eight weeks of survival the β-secretase seemed to be accumulated in some areas of the pyramidal layer while other areas showed less amount of β-secretase (FIG. 4B).

Sham operated animals did not reveal any significant immunostaining when either Aβ antibody or β-secretase antibody was used.

Immunohistochemistry of Cortex:

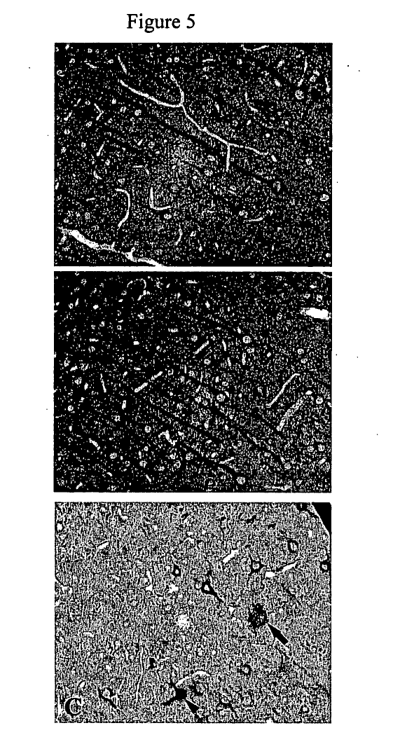

Sham operated animals showed PAPP staining in the cytoplasm of neurons in parietal cortex layer II-IV and in the dendrites (FIG. 5A). At four weeks of recovery staining of βAPP could be seen.in parietal cortex where the cytoplasm of neurons were stained as well as several of the dendrites (FIG. 5B). At ten weeks of recovery there was more βAPP accumulated in the cytoplasm and less in dendrites. Some neurons showed immunostaining in the whole cell. Staining could also be seen outside the neurons that seemed to be extracellular deposits (FIG. 5C).

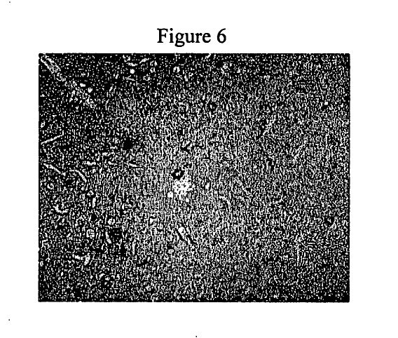

Weak staining of Aβ was seen as granules in the cytoplasm of many neurons in parietal cortex at four weeks post ischemia (FIG. 6). The staining at ten weeks post ischemia looked almost similar, although there might be fewer immunostained neurons.

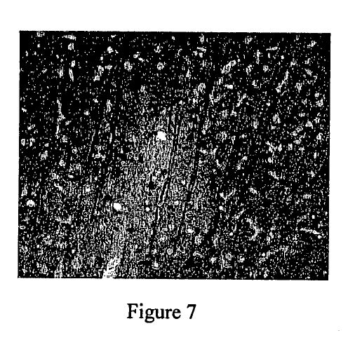

Immunolabelling with the β-secretase antibody was seen in the cytoplasm in some of the neurons in the parietal cortex as a punctate pattern at four weeks of recovery. Some neurons showed staining in the whole cell. Structures that seemed to be dendrites were also immunostained (FIG. 7). At eight weeks of recovery staining was less intensive, and the staining seemed to have a more truncated pattern of structures that appeared to be the dendrites.

Comparative Immunohistochemical Studies of Aged and Young Animals

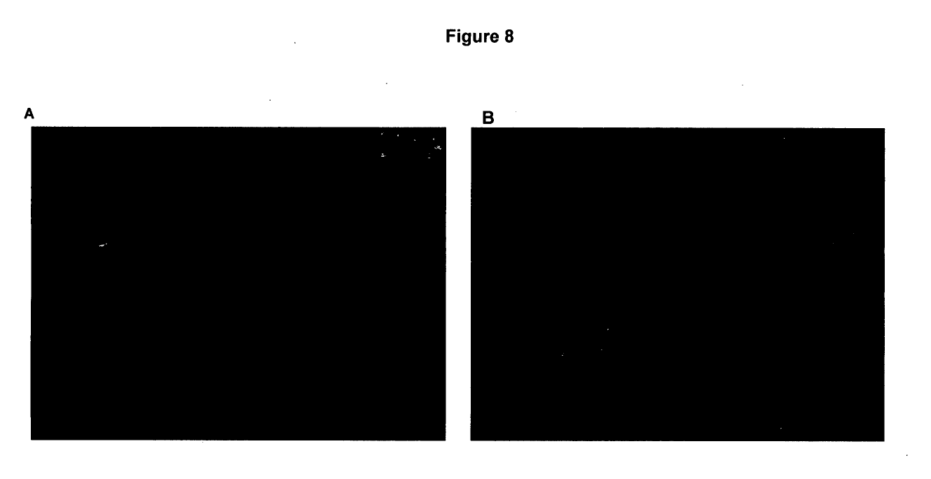

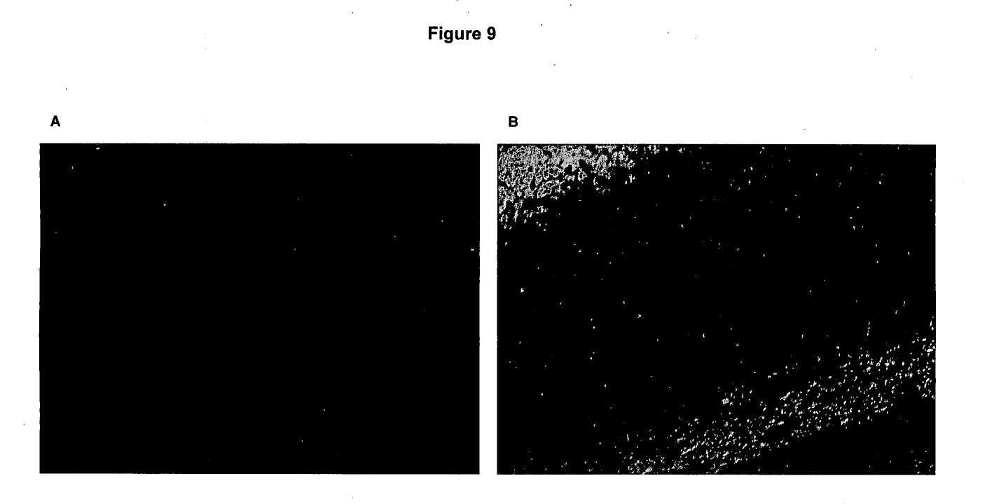

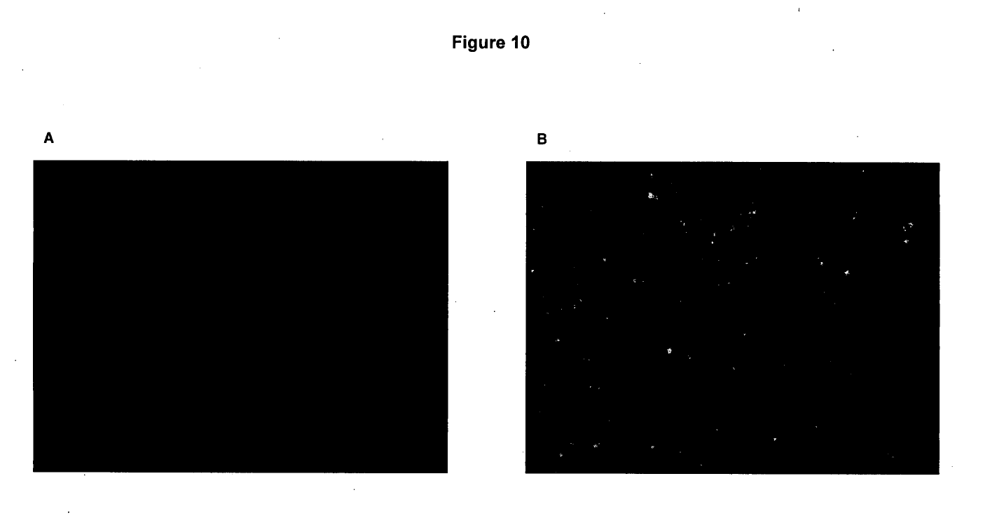

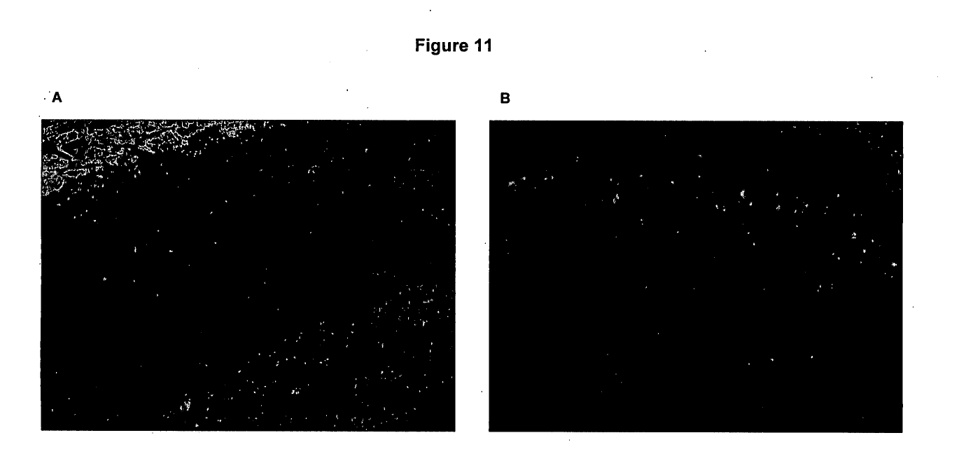

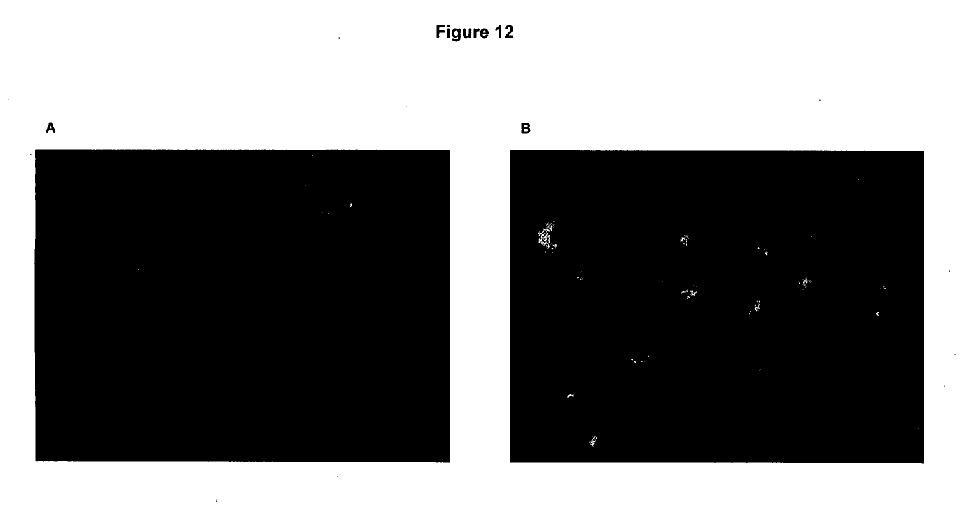

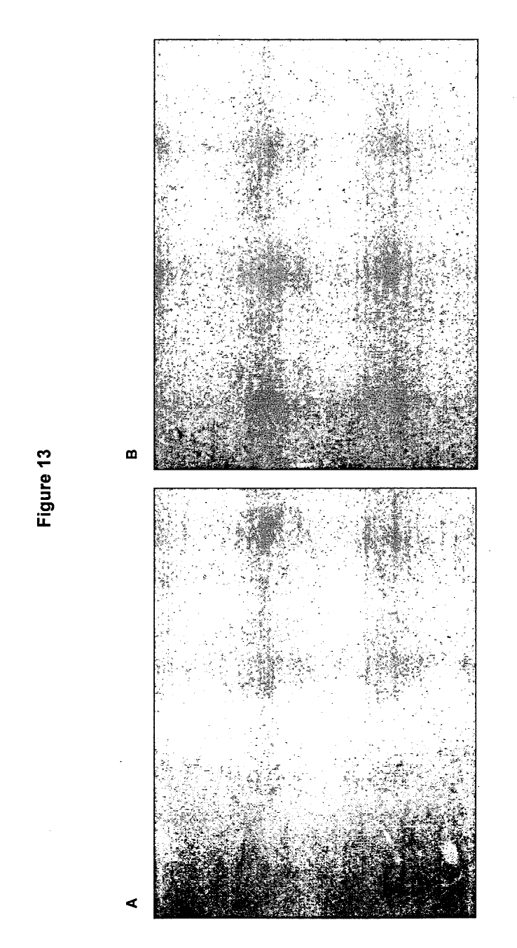

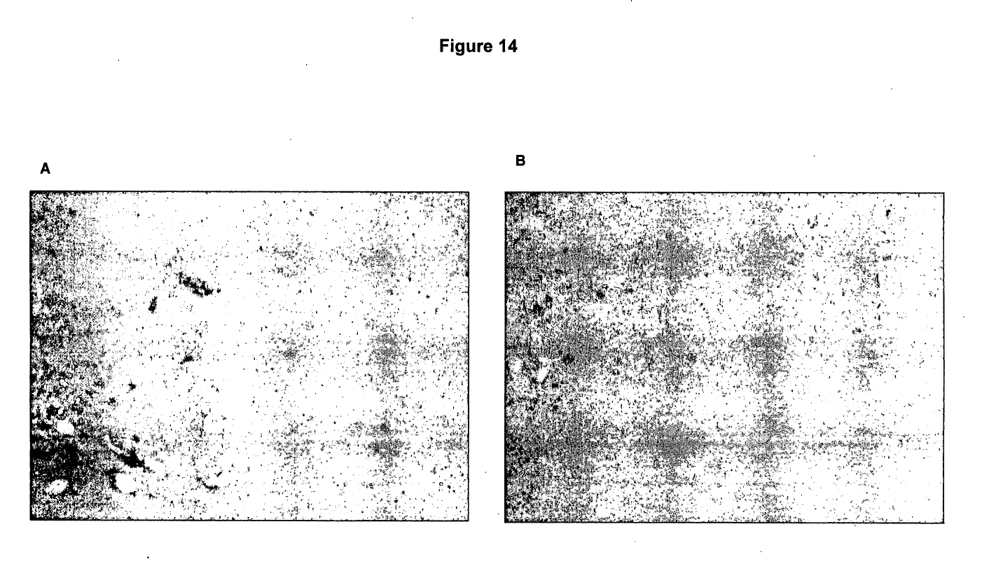

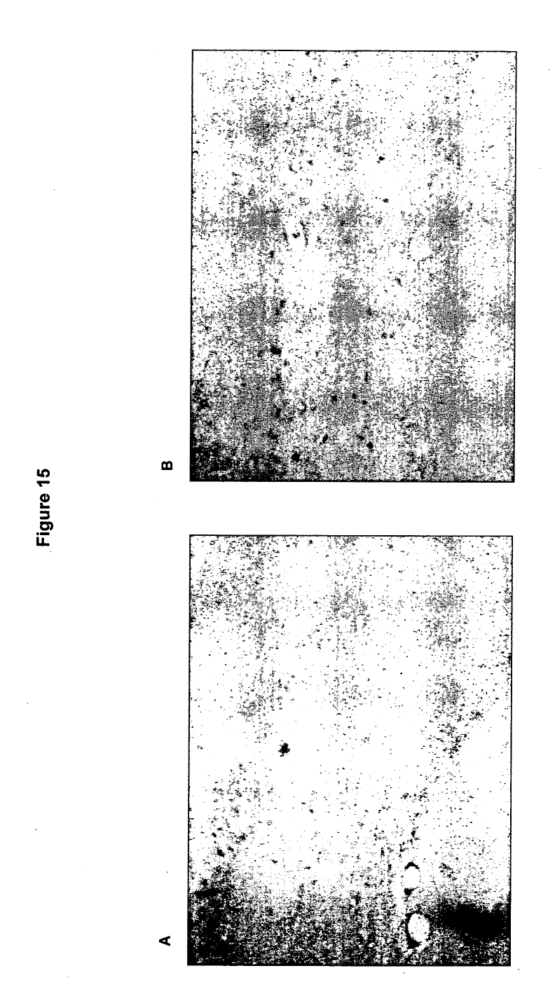

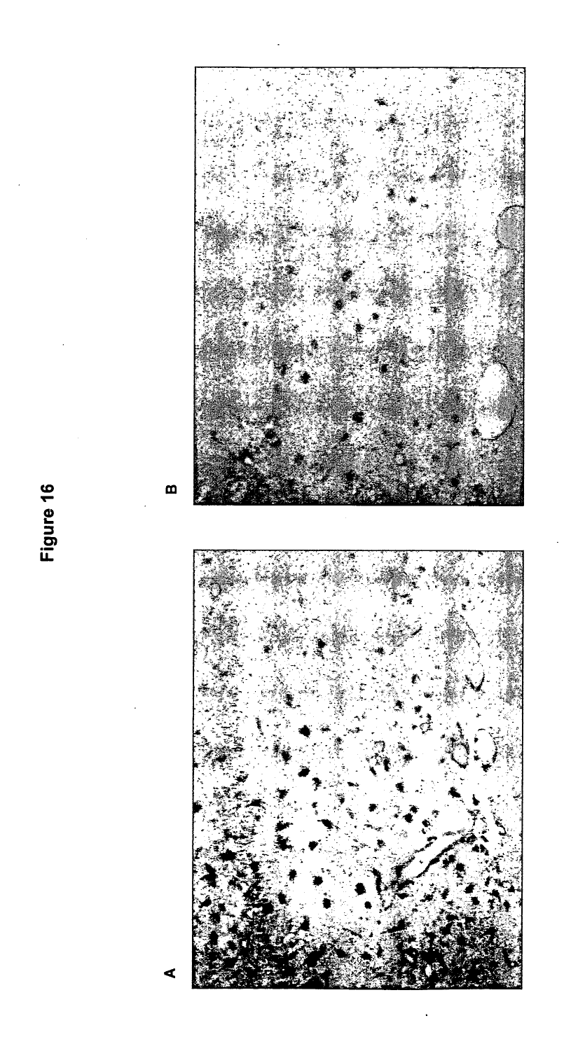

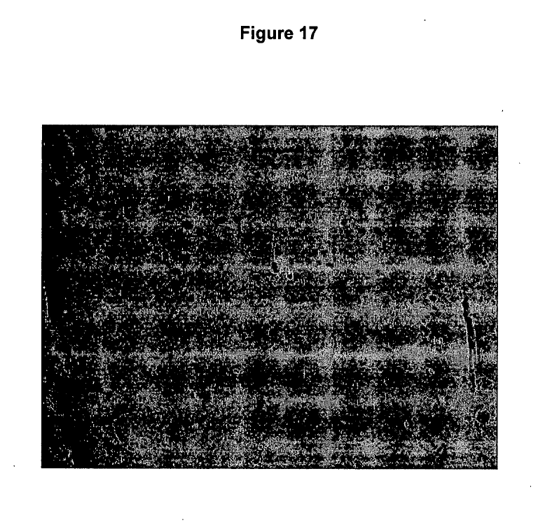

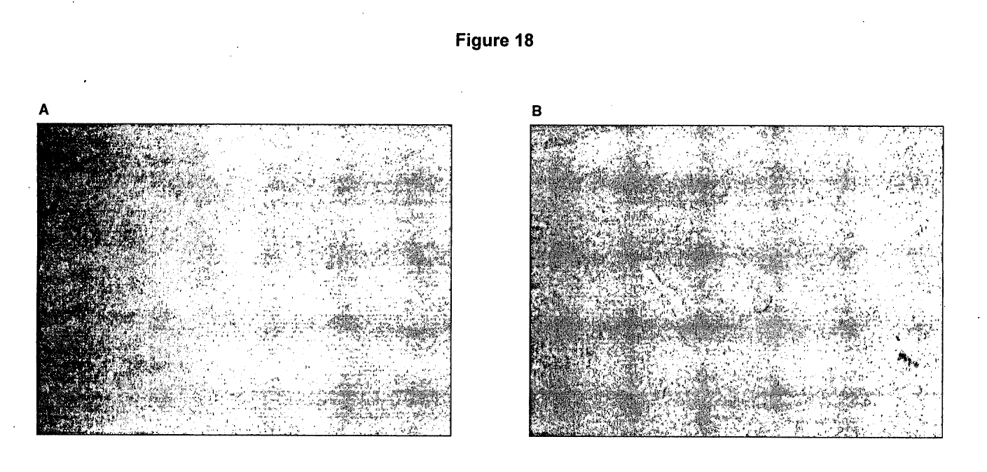

Our comparative studies (FIGS. 8-18) of various AD-associated polypeptides in young and aged animals further validate the applicability of the subject AD animal models. Our results show that in older animals, Aβ deposition is much more severe than in young animals. By contrast, Aβ deposits tend to dissolve at later recovery time points in similarly treated young animals. In addition, βAPP is expressed at a lower level in aged animals than young animals after the ischemic treatment.

TABLE 1
Summary of level of proteins expressed in young or aged rats at 4 or 10
weeks recovery after 15 minutes of ischemia. The protein level is
determined by immunostaining using antibodies directed to β-APP,
β-secretase, or Aβ peptide.

| | |
|---|---|
| Young | Aged |

| Molecule | 4 week | 10 week | 4 week | 10 week |
|---|---|---|---|---|
| β-APP | ++ | ++ | + | + |
| β-secretase | ++ | + | + | + |
| Aβ peptide | ++ | + | +++ | +++ |

Discussion:

Our study demonstrates that prolonged forebrain ischemia in animals can induce pathological conditions that are characteristic of AD. The animal model so generated provides a useful tool for AD research and drug development.

Neurodegenerative Changes During Ischemia-Reperfusion

Following an acute neuronal degeneration in selective vulnerable brain regions, for example hippocampal CA1 region, there are protracted degenerative changes lasting long time after a transient ischemic insult (Lin, et al. (1998) Acta Neuropathol 95:511-523; Lin, et al. (1999) Acta Neuropathol 97:359-368). These chronic brain pathological changes share some similarity with AD. Pluta et al. describes a cardiac arrest model and its relevance in clinical studies (Pluta, et al. (2000) Ann NY Acad Sci 903:324-334). Pluta et al. reports the correlation between apolipoprotein E (ApoE) and Aβ. Specifically, at six months of survival immunostaining of Aβ in glial cells in hippocampus was observed and after one year of survival there was extracellular deposits of Aβ in the entorhinal cortex.

Amyloid Precursor Protein Changes During Ischemia-Reperfusion:

Our study revealed granular staining in the strata oriens and strata radiatum at about four weeks of recovery. At about ten weeks the granules seen in strata oriens and radiatum were less than at about four weeks, and the staining were more concentrated to the pyramidal layer as intensively stained deposits of βAPP. Also neuronal remnants were diffusely stained. Pluta showed that ApoE was diffusely stained in hippocampus in neuronal remnants and suggested that formation of ApoE-Aβ complexes, which is known to exist in the brain of AD patients. These complexes adhere to the neuronal remnants after neuronal death and produce diffuse amyloid plaques. It has also been shown that perivascular deposits of ApoE co-localize with epitopes on βAPP. Kida, et al. (1995) Brian Res 674:341-346. These results suggest that βAPP might be involved in the production of diffuse amyloid plaque with in the pyramidal layer.

β-Secretase Changes During Ischemia-Reperfusion:

At about four weeks of recovery the β-secretase could be seen as a punctate pattern in the pyramidal layer of CA1. Approximately at eight weeks we observed accumulation of β-secretase in some areas of the pyramidal layer. This accumulation correlates with the accumulation of βAPP.

Aβ Changes During Ischemia-Reperfusion:

At about four weeks of recovery striking immunoreactivity of both β-secretase and Aβ in the CA1 of hippocampus was seen. At about ten weeks of recovery the staining was less intensive suggesting that the Aβ probably was degraded. In an immunotheraphy model where antibodies against Aβ were injected into the rat brain, decreased number of Aβ deposits and increased levels of microglia among the remaining plaques were observed, suggesting that microglia was involved in the reduction of the plaque (Bacskai, et al. (2001) Nature Med 7:369-372).

We claim:

1. A method of generating an animal model exhibiting a pathological condition of Alzheimer's disease, comprising:
(a) inducing a transient forebrain ischemia in the animal; and
(b) allowing the animal to recover from the ischemic induction for a sufficient amount of time so that a pathological condition of Alzheimer's disease is exhibited.

2. The method of claim 1, wherein the animal is selected from the group consisting of mammal, primate, and rodent.

3. The method of claim 1, wherein the animal is rat.

4. The method of claim 1, wherein the animal is selected from the group consisting of mouse, guinea pig, dog, cat, rabbit, pig, chimpanzee, and monkey.

5. The method of claim 1, wherein the pathological condition is characterized by differential expression of an Alzheimer's disease-associated gene or polypeptide.

6. The method of claim 5, wherein the Alzheimer's disease-associated polypeptide is a beta-amyloid peptide.

7. The method of claim 5, wherein the Alzheimer's disease-associated gene or polypeptide is beta-secretase.

8. The method of claim 5, wherein the differential expression of an Alzheimer's disease-associated polypeptide is detected by an immunoassay.

9. The method of claim 5, wherein the differential expression of an Alzheimer's disease-associated polypeptide is detected by a hybridization assay.

10. The method of claim 1, wherein the animal is allowed to recover for at least about 2 weeks.

11. The method of claim 1, wherein the animal is allowed to recover for at least about 4 weeks.

12. The method of claim 1, wherein the animal is allowed to recover for about 2 to about 10 weeks.

13. The method of claim 1, wherein the animal is allowed to recover for about 4 to about 10 weeks.

14. The method of claim 1, wherein the ischemic induction lasts for more than 10 minutes.

15. The method of claim 1, wherein the ischemic induction lasts for about 15 minutes to about 20 minutes.

16. An animal model generated by the method of claim 1.

17. A method of developing a modulator of pathogenesis of Alzheimer's disease, comprising:
(a) administering a candidate modulator to a test animal model generated by a method comprising (i) inducing a transient and reversible forebrain ischemia in the animal; and (ii) allowing the animal to recover from the ischemic induction for a sufficient amount of time so that a pathological condition of Alzheimer's disease is exhibited; and
(b) detecting a change in the pathological condition in the test animal model of (a) relative to a control.

18. The method of claim 17, wherein the modulator ameliorates the pathological condition of Alzheimer's disease.

19. The method of claim 17, wherein the modulator advances the pathological condition of Alzheimer's disease.

20. The method of claim 17, where the pathological condition is characterized by differential expression of an Alzheimer's disease-associated gene or polypeptide.

21. The method of claim 20, where the Alzheimer's disease-associated polypeptide is a beta-amyloid peptide.

22. The method of claim 20, where the Alzheimer's disease-associated gene or polypeptide is beta-secretase.

23. The method of claim 20, wherein the differential expression of an Alzheimer's disease-associated polypeptide is detected by an immunoassay.

24. The method of claim 20, wherein the differential expression of an Alzheimer's disease-associated gene is detected by a hybridization assay.

25. The method of claim 17, where the pathological condition is selected from the group consisting of beta-amyloid plaque formation, plaque-induced mononuclear phagocyte activation, plaque-induced mononuclear phagocyte neurotoxicity, and neuronal loss within the brain.

26. The method of claim 17, wherein the candidate modulator is selected from the group consisting of an antisense oligonucleotide, a ribozyme, a ribozyme derivative, an antibody, a liposome, a small interfering RNA, a small molecule and an inorganic compound.

27. The method of claim 17, wherein the test animal model is selected from the group consisting of mammal, primate, and rodent.

28. The method of claim 17, wherein the animal is selected from the group consisting of rat, mouse, guinea pig, dog, cat, rabbit, pig, chimpanzee, and monkey.

29. The method of claim 17, wherein the control is an animal to which the candidate modulator is not administered or is administered at a lower dose, or is administered for a shorter period of time, relative to the test animal model.

30. The method of claim 17, wherein the control also exhibits the pathological condition of Alzheimer's disease.

31. The method of claim 30, wherein the control is generated by (i) inducing a transient forebrain ischemia in the animal; and (ii) allowing the animal to recover from the ischemic induction for a sufficient amount of time so that a pathological condition of Alzheimer's disease is exhibited

32. The method of claim 17 or 31, wherein the ischemic induction lasts more than 10 minutes.

33. The method of claim 17 or 31, wherein the ischemic induction lasts about 15 to about 20 minutes.

34. The method of claim 17, wherein the animal is allowed to recover for at least about 2 weeks.

35. The method of claim 17, wherein the animal is allowed to recover for at least about 4 weeks.

36. The method of claim 17, wherein the animal is allowed to recover for about 2 to about 10 weeks.

37. The method of claim 17, wherein the animal is allowed to recover for about 4 to about 10 weeks.

38. The method of claim 17, where the candidate modulator is administered to the test animal model intravenously.

39. The method of claim 17, where the candidate modulator is administered to the test animal model subcutaneously, intramuscularly, intraperitoneally, intradermally, orally, intranasally, or intrapulmonarily.

40. A method of developing a modulator of an Alzheimer's disease-associated gene or protein, comprising:
(a) contacting a candidate modulator with an Alzheimer's disease-associated gene or protein that is contained in a test biological sample derived from an animal model, wherein the animal model is generated by a method comprising:
(i) inducing a transient forebrain ischemia in the animal; and
(ii) allowing the animal to recover from the ischemic induction for a sufficient amount of time so that a pathological condition of Alzheimer's disease is exhibited; and
(b) detecting an alteration in expression of the Alzheimer's disease-associated gene or protein, or an alteration in activity of the protein of step (a), relative to a control sample.

41. The method of claim 40, wherein the modulator ameliorates the pathological condition of Alzheimer's disease.

42. The method of claim 40, wherein the modulator advances the pathological condition of Alzheimer's disease.

43. The method of claim 40, where the pathological condition is characterized by differential expression of an Alzheimer's disease-associated gene or polypeptide.

44. The method of claim 40, where the Alzheimer's disease-associated polypeptide is a beta-amyloid peptide.

45. The method of claim 40, where the Alzheimer's disease-associated gene or polypeptide is beta-secretase.

46. The method of claim 40, where the pathological condition is selected from the group consisting of beta-amyloid plaque formation, plaque-induced mononuclear phagocyte activation, plaque-induced mononuclear phagocyte neurotoxicity, and neuronal loss within the brain.

47. The method of claim 40, wherein the alteration in expression of the Alzheimer's disease-associated gene is assayed by a hybridization assay.

48. The method of claim 40, wherein the alteration in expression of the Alzheimer's disease-associated protein is detected by an immunoassay.

49. The method of claim 40, wherein the candidate modulator is selected from the group consisting of an antisense oligonucleotide, a ribozyme, a ribozyme derivative, an antibody, a liposome, a small interfering RNA, a small molecule and an inorganic compound.

50. The method of claim 40, wherein the test animal model is selected from the group consisting of mammal, primate, and rodent.

51. The method of claim 40, wherein the animal is selected from the group consisting of rat, mouse, guinea pig, dog, cat, rabbit, pig, chimpanzee, and monkey.

52. The method of claim 40, wherein the ischemic induction lasts more than 10 minutes.

53. The method of claim 40, wherein the ischemic induction lasts about 15 to about 20 minutes.

54. The method of claim 1, wherein the animal is an aged animal.
